(19) United States
(12) Patent Application Publication
(10) Pub. No.: US 2026/0314153 A1
(43) Pub. Date: Oct. 8, 2026

(54) LIGHT IRRADIATION DEVICE AND LIGHT IRRADIATION APPARATUS

(71) Applicant: INNOLUX CORPORATION, Miao-Li County (TW)

(72) Inventors: Chun-Yu CHEN, Miao-Li County (TW); Chun-Kai LEE, Miao-Li County (TW); Cheng-Hsu CHOU, Miao-Li County (TW)

(21) Appl. No.: 19/573,814

(22) Filed: Mar. 20, 2026

(30) Foreign Application Priority Data
Apr. 7, 2025 (CN) 202510423610.0

(51) Int. Cl. H10H 29/85; H10K 59/80

(52) U.S. Cl. CPC H10H 29/8582; H10H 29/8581; H10K 59/8794

(57) ABSTRACT

A light irradiation device includes a substrate, a light-emitting unit, a package layer and a thermal conductive layer. The light-emitting unit is disposed on the substrate. The package layer is disposed at one side of the light-emitting unit away from the substrate. The thermal conductive layer is disposed at one side of the package layer away from the light-emitting unit. The light transmittance of the thermal conductive layer ranges from 80% to 99%. The light-emitting unit is configured to emit a light having a wavelength between 650 nm and 830 nm, and the light intensity range of the light is set from 1 mW/cm² to 200 mW/cm².

### CROSS REFERENCE TO RELATED APPLICATIONS

This Non-provisional application claims priority under 35 U.S.C. § 119(a) on Patent Application No(s). 202510423610.0 filed in China on Apr. 7, 2025, the entire contents of which are hereby incorporated by reference.

### BACKGROUND

### Technology Field

The present disclosure relates to a light irradiation device and a light irradiation apparatus.

### Description of Related Art

Many types of light irradiation devices are used in medical-related fields, such as phototherapy devices, laser acupuncture devices or the likes. In recent years, the light sources of these light irradiation devices have gradually adopted light-emitting diodes (LEDs). However, the electroluminescent efficiency of LEDs is not 100%, so when they are driven to emit light, they also generate heat energy. In addition, to increase the light dose, the driving current of the LED must be increased, which causes the increase of surface temperature of LED. If these light irradiation devices are used in skin-mounted applications, the heat energy generated by the LED may pose a risk of burns to the user's skin. To avoid this risk of burns, the driving current of the LED is usually reduced to lower the temperature thereof, but this also reduces the light intensity of the light irradiation device. In this case, if the light dose is to be increased, the light exposure time must be extended to compensate the light dose.

### SUMMARY

This disclosure provides a light irradiation device and a light irradiation apparatus that can effectively increase the light intensity while avoiding the risk of burns.

A light irradiation device of this disclosure includes a substrate, a light-emitting unit, a package layer, and a thermal conductive layer. The light-emitting unit is disposed on the substrate, the package layer is disposed on the light-emitting unit, and the thermal conductive layer is disposed on the package layer. The light transmittance of the thermal conductive layer ranges from 80% to 99%. The light-emitting unit is configured to emit a light having a wavelength between 650 nm and 830 nm, and a light intensity range of the light is set from 1 mW/cm² to 200 mW/cm².

This disclosure also provides a light irradiation apparatus including a control device and the above-mentioned light irradiation device.

### BRIEF DESCRIPTION OF THE DRAWINGS

The disclosure will become more fully understood from the detailed description and accompanying drawings, which are given for illustration only, and thus are not limitative of the present disclosure, and wherein:

FIG. 1 is a schematic sectional view of a light irradiation device according to a first embodiment of this disclosure;

FIG. 2A is a schematic diagram showing a simulation experiment in which the light irradiation device of FIG. 1 is applied to human skin;

FIG. 2B is a schematic top view of the light irradiation device during the simulation experiment shown in FIG. 2A;

FIG. 3 is a schematic sectional view of a light irradiation device according to a second embodiment of this disclosure;

FIG. 4 is a schematic sectional view of a light irradiation device according to a third embodiment of this disclosure;

FIG. 5A is a schematic perspective view of the light irradiation apparatus according to an embodiment of this disclosure;

FIG. 5B is a schematic diagram of the light irradiation apparatus of FIG. 5A in use;

FIG. 6A is a schematic perspective view of a light irradiation apparatus according to another embodiment of this disclosure; and

FIG. 6B is a schematic diagram of the light irradiation apparatus of FIG. 6A in use.

### DETAILED DESCRIPTION OF THE DISCLOSURE

The present disclosure will be apparent from the following detailed description, which proceeds with reference to the accompanying drawings, wherein the same references relate to the same elements.

To be noted, the features of several different embodiments may be replaced, reorganized, or combined to create other embodiments without departing from the spirit of the present disclosure.

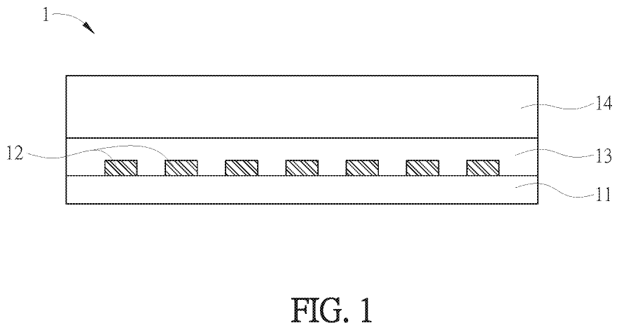

FIG. 1 is a schematic sectional view of a light irradiation device 1 according to a first embodiment of this disclosure.

As shown in FIG. 1, the light irradiation device 1 of this embodiment includes a substrate 11, a light-emitting unit 12, a package layer 13, and a thermal conductive layer 14. In this embodiment, the light irradiation device 1 may be, for example but not limited to, a phototherapy device or a laser acupuncture device. The phototherapy device, for example, can irradiate a wound site with a light to reduce inflammation, speed wound healing, promote collagen synthesis, and accelerate angiogenesis. The laser acupuncture device, for example, can stimulate traditional acupuncture points with a light to provide energy for stimulating the patient body, thereby achieving the effects of traditional acupuncture.

The substrate 11 may be, for example, a rigid or flexible substrate, and may include, for example but not limited to, transparent or opaque organic and/or inorganic materials. The organic material may include, for example but not limited to, polyimide (PI), fiber-reinforced plastic (FRP), polyethylene terephthalate (PET), cycloolefin polymers (COP), or any of other known suitable materials or combinations of the above materials. The inorganic material may include, for example but not limited to, glass, quartz, sapphire, or ceramics. The material of the substrate 11 of this embodiment is polyimide, for example, so that the substrate 11 is a flexible substrate. In addition, the substrate 11 can be a substrate including a circuit layer (not shown) electrically connected to the light-emitting unit 12. This circuit layer, for example, includes various passive and/or active components, such as resistors, capacitors, inductors, diodes, metal oxide semiconductor field effect transistors (MOSFETs), complementary metal oxide semiconductor (CMOS) transistors, bipolar junction transistors (BJTs), laterally diffused metal oxide semiconductor (LDMOS) transistors, high-power metal oxide semiconductor transistors, thin film transistors (TFTs), or other types of transistors. In addition, the substrate 11 can be a driving substrate that drives the light-emitting unit 12 to emit light, such as a CMOS substrate, a liquid crystal on silicon (LCOS) substrate, a thin-film transistor (TFT) substrate, or other circuit substrates with working circuits. In addition, the size of the substrate 11 can be, for example but not limited to, the size of a coin or larger, and its shape can be, for example but not limited to, circular, rectangular, elliptical, etc.

The light-emitting unit 12 is disposed on the substrate 11. In this embodiment, the light-emitting unit 12 includes, for example, a plurality of light-emitting diode (LED) elements, such as organic light-emitting diodes (OLEDs), inorganic LEDs (e.g. LEDs, mini LEDs, micro LEDs, QLEDs), or other suitable materials, or combinations thereof. In this case, the circuit elements in the circuit layer of the substrate 11 can be used to control and drive the light-emitting unit 12, so that the light-emitting unit 12 can emit a light. The emitted light can be, for example, a visible light or an invisible light. In some embodiments, the emitted light can be, for example, red light and/or infrared light, the wavelength thereof can be, for example, between 650 nm and 830 nm, and the light intensity range thereof is set from 1 mW/cm² to 200 mW/cm². For example, the light-emitting unit 12 can include one LED element or multiple different LED elements, such as a red LED (for emitting red light with a wavelength of approximately 620-750 nm), an infrared LED (for emitting infrared light with a wavelength of approximately 800-950 nm), etc. Therefore, the light-emitting unit 12 can be driven to emit a light with a wavelength between 650 nm and 830 nm. In addition, by applying different driving currents to the light-emitting unit 12, the light intensity range of the emitted lights can be set to 1-200 mW/cm². In this case, the applied driving current is, for example but not limited to, between 10 mA and 10 A.

The package layer 13 is disposed on the light-emitting unit 12. In one embodiment, the package layer 13 is located on the side of the light-emitting unit 12 opposite to the bonding interface of the substrate 11 and the light-emitting unit 12. In this embodiment, the package layer 13 can be formed on the light-emitting unit 12 and the substrate 11, and surrounding the light-emitting unit 12. The light emitted by the light-emitting unit 12 can be emitted through the package layer 13. The material of the package layer 13 can be, for example, a material with a low water vapor transmission rate (WVTR), such as, for example but not limited to, SiOₓN_{y}, SiOₓ, SiNₓ, SiO₂, ZnO, Al₂O₃, or any combination thereof. The package layer 13 can be formed by using a CVD process or an ALD process. In some embodiments, the package layer 13 can be a single layer or a multilayer structure. Therefore, the package layer 13 can be used to protect the multiple LEDs of the light-emitting unit 12 from external moisture.

The thermal conductive layer 14 is disposed on the package layer 13. In one embodiment, the thermal conductive layer 14 is located on the side of the package layer 13 opposite to the side thereof facing the light-emitting unit 12. In one embodiment, the side of the thermal conductive layer 14 opposite to the side thereof facing the light-emitting unit 12 can be used to contact the body surface. The light transmittance of the thermal conductive layer 14 is between 80% and 99%. In some embodiments, the light transmittance of the thermal conductive layer 14 is between 90% and 99%. In some embodiments, the thickness of the thermal conductive layer 14 is, for example, between 0.05 mm and 5 mm, and the thermal conductivity of the thermal conductive layer 14 is, for example, between 0.2 W/m-K and 4 W/m-K. In some embodiments, the thickness of the thermal conductive layer 14 is, for example, between 0.3 mm and 2 mm, and the thermal conductivity of the thermal conductive layer 14 is, for example but not limited to, between 0.6 W/m-K and 2 W/m-K. In this embodiment, the configuration of the thermal conductive layer 14 can provide a limited thermal conductivity. Specifically, the thermal conductivity of the thermal conductive layer 14 is significantly lower than that of typical high-performance thermal conductive materials. For example, the thermal conductivity of copper, which is generally considered a high-performance thermal conductive material, is approximately 401 W/m-K. Therefore, the thermal conductive layer 14 only partially conducts heat energy from the side thereof adjacent to the package layer 13 and the light-emitting unit 12 to the side thereof away from the package layer 13. In one embodiment, the side of the thermal conductive layer 14 away from the package layer 13 can be the side configured to contact the body surface. This design allows the temperature of the side of the thermal conductive layer 14 away from the package layer 13 to be lower than the temperature of the side of the thermal conductive layer 14 adjacent to the package layer 13. In practice, the side of the thermal conductive layer 14 away from the package layer 13 can be directly attached to the user's skin. Therefore, the limited thermal conductivity of the thermal conductive layer 14 allows the temperature of the side of the thermal conductive layer 14 attached to the user's skin to be controlled to be above body temperature but below a temperature that could cause skin burns (e.g., 45° C.). In some embodiments, the thermal conductivity can be measured, for example, by using a thermal conductivity analyzer. In some embodiments, for example, the intensity of the light emitted by the light irradiation device and the intensity of the light emitted by a reference light irradiation device (without the thermal conductive layer 14) can be measured separately, and the two light intensities can be compared to each other to obtain the light transmittance. The light intensity can be measured, for example, using an illuminometer.

In addition, in this embodiment, the thermal conductive layer 14 may, for example, include a biocompatible material. Therefore, in one embodiment, the thermal conductive layer 14 can be suitable for long-term attachment to a body surface, such as the skin surface. The material of the thermal conductive layer 14 may be, for example but not limited to, a biocompatible hydrogel material or silicone material, or a combination thereof, or any material that can be used as a dressing for the affected area. In one embodiment, the thermal conductive layer 14 may be a single-layer structure or a multi-layer structure (e.g. a double-layer structure). For example, the thermal conductive layer 14 may be, for example, a single-layer structure including the hydrogel material or silicone material, a single-layer structure including a mixture of the hydrogel material or silicone material and a pressure-sensitive adhesive (PSA), a double-layer structure including a layer of hydrogel material or silicone material and a layer of pressure-sensitive adhesive, or a double-layer structure including a layer of hydrogel material or silicone material and a layer of the mixture of the hydrogel material or silicone material and the pressure-sensitive adhesive.

In another embodiment, the thermal conductive layer 14 may include a pharmaceutical composition, such as, for example but not limited to, a wound dressing, a percutaneous absorption drug, or a combination thereof. The wound dressing may be, for example, any substance that can act on the human body or a material that can accelerate wound healing, and the percutaneous absorption drug may be, for example, any material that can be absorbed through the skin. For example, the pharmaceutical composition may be, for example but not limited to, chemically synthesized medicinal materials or natural medicinal materials, such as pain treatment herbs (e.g. peppermint oil, camphor, eucalyptus oil, capsaicin, ginger extract, mugwort, angelica dahurica, etc.), or herbs that increase blood circulation (e.g. safflower, salvia miltiorrhiza, cassia twig, frankincense, myrrh, etc.).

In addition, the light irradiation device 1 may further include a release film (not shown) that is removably attached to the side of the thermal conductive layer 14 away from the package layer 13. In one embodiment, the side of the thermal conductive layer 14 away from the package layer 13 may be the side of the thermal conductive layer 14 that contacts the body surface. In practice, the release film protects the thermal conductive layer 14 when not in use. When using the light irradiation device 1, the release film can be peeled off from one side of the thermal conductive layer 14 before the thermal conductive layer 14 of the light irradiation device 1 is attached to the affected area for subsequent treatment, such as phototherapy or laser acupuncture.

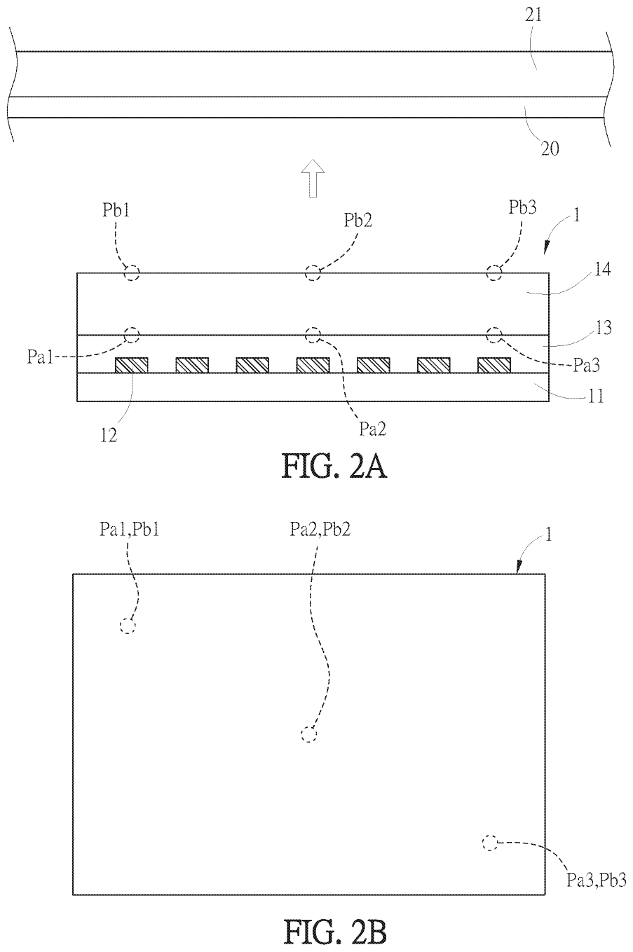

A simulation experiment of applying the light irradiation device 1 to human skin will be discussed hereinafter with reference to FIG. 2A and FIG. 2B.

In this simulation experiment, the shape of the light irradiation device 1 is, for example, rectangular (as shown in FIG. 2B). First, three thermometer probes are placed between the package layer 13 and the thermal conductive layer 14, with their placement positions Pa1, Pa2 and Pa3 as shown in FIG. 2A and FIG. 2B. Next, three additional thermometer probes are placed on the side of the thermal conductive layer 14 away from the package layer 13, with their placement positions Pb1, Pb2 and Pb3 as shown in FIG. 2A and FIG. 2B. Then, the thermal conductive layer 14 of the light irradiation device 1 is attached to one side of the artificial skin 20, and a heating plate 21 is placed on the other side of the artificial skin 20. The heating plate 21 is set to maintain a heated state in the temperature of 34° C.

As mentioned above, in one example, the thermal conductive layer 14 is a single-layer structure with a thickness of 0.6 mm. After the thermal conductive layer 14 is attached to the artificial skin 20, a constant current of 1.5 A (approximately 3.8V) is applied to the light-emitting unit 12 to cause it to emit light and generate heat. The temperature data at positions Pa1, Pa2, Pa3, Pb1, Pb2, and Pb3 are continuously measured for approximately 20 minutes. The maximum temperatures at these positions are summarized in Table 1 below.

a

TABLE 1

| | Position | | | | | |
|---|---|---|---|---|---|---|
| | Pa1 | Pa2 | Pa3 | Pb1 | Pb2 | Pb3 |
| Max. | 44.9 | 55.2 | 46.3 | 39.8 | 46.2 | 41.5 |
| temperature | | | | | | |
| (° C.) | | | | | | |

In another example, the thermal conductive layer 14 is a single-layer structure with a thickness of 1.2 mm. After the thermal conductive layer 14 is attached to the artificial skin 20, a constant current of 1.5 A (approximately 3.8V) is applied to the light-emitting unit 12 to cause it to emit light and generate heat. The temperature data at positions Pa1, Pa2, Pa3, Pb1, Pb2, and Pb3 are continuously measured for approximately 20 minutes. The maximum temperatures at these positions are summarized in Table 2 below.

TABLE 2

| | Position | | | | | |
|---|---|---|---|---|---|---|
| | Pa1 | Pa2 | Pa3 | Pb1 | Pb2 | Pb3 |
| Max. | 47.2 | 56.8 | 47.7 | 40.0 | 44.5 | 43.9 |
| temperature | | | | | | |
| (° C.) | | | | | | |

In another example, the thermal conductive layer 14 is a single-layer structure with a thickness of 2.0 mm. After the thermal conductive layer 14 is attached to the artificial skin 20, a constant current of 1.5 A (approximately 3.8V) is applied to the light-emitting unit 12 to cause it to emit light and generate heat. The temperature data at positions Pa1, Pa2, Pa3, Pb1, Pb2, and Pb3 are continuously measured for approximately 20 minutes. The maximum temperatures at these positions are summarized in Table 3 below.

TABLE 3

| | Position | | | | | |
|---|---|---|---|---|---|---|
| | Pa1 | Pa2 | Pa3 | Pb1 | Pb2 | Pb3 |
| Max. | 48.3 | 58.3 | 48.3 | 39.3 | 43.1 | 42.0 |
| temperature | | | | | | |
| (° C.) | | | | | | |

In another example, the thermal conductive layer 14 is a double-layer structure having a first layer with a thickness of 1.2 mm and a second layer with a thickness of 2.0 mm. After the thermal conductive layer 14 is attached to the artificial skin 20, a constant current of 1.5 A (approximately 3.8V) is applied to the light-emitting unit 12 to cause it to emit light and generate heat. The temperature data at positions Pa1, Pa2, Pa3, Pb1, Pb2, and Pb3 are continuously measured for approximately 20 minutes. The maximum temperatures at these positions are summarized in Table 4 below.

TABLE 4

| | Position | | | | | |
|---|---|---|---|---|---|---|
| | Pa1 | Pa2 | Pa3 | Pb1 | Pb2 | Pb3 |
| Max. | 48.5 | 60.5 | 49.5 | 40.6 | 46.9 | 39.0 |
| temperature | | | | | | |
| (° C.) | | | | | | |

In another example, the thermal conductive layer 14 is a double-layer structure having a first layer with a thickness of 2.0 mm and a second layer with a thickness of 2.0 mm. After the thermal conductive layer 14 is attached to the artificial skin 20, a constant current of 1.5 A (approximately 3.8V) is applied to the light-emitting unit 12 to cause it to emit light and generate heat. The temperature data at positions Pa1, Pa2, Pa3, Pb1, Pb2, and Pb3 are continuously measured for approximately 20 minutes. The maximum temperatures at these positions are summarized in Table 5 below.

TABLE 5

| | Position | | | | | |
|---|---|---|---|---|---|---|
| | Pa1 | Pa2 | Pa3 | Pb1 | Pb2 | Pb3 |
| Max. | 49.8 | 60.5 | 51.7 | 40.5 | 44.3 | 38.4 |
| temperature | | | | | | |
| (° C.) | | | | | | |

Based on the above examples, it is obvious that the light irradiation device 1 of this embodiment can partially block the heat energy generated by the light-emitting unit 12 by virtue of the limited heat conductivity of the thermal conductive layer 14, thereby reducing the temperature of the side of the thermal conductive layer 14 attached to the user's skin (e.g. positions Pb1, Pb2, and Pb3). For example, the temperature of the side of the thermal conductive layer 14 attached to the user's skin can be controlled to be higher than the human body temperature but lower than the temperature that may cause burns to the skin (e.g. below 45° C.), thereby avoiding the risk of burns. In addition, the above examples also show the following characteristics. When the thickness of the thermal conductive layer 14 is thicker, the heat energy generated by the light-emitting unit 12 is more difficult to discharge, resulting in a higher temperature between the package layer 13 and the thermal conductive layer 14 (positions Pa1, Pa2, and Pa3). In addition, when the thickness of the thermal conductive layer 14 is thicker, the heat energy generated by the light-emitting unit 12 is transferred to the surface of the thermal conductive layer 14 away from the package layer 13 (positions Pb1, Pb2, and Pb3) more slowly.

In one example, under the critical condition that the temperature of the attachment surface does not exceed 45° C., the driving current of the light irradiation device must be controlled below approximately 1.0 A, and the intensity of the emitted light is approximately 14.1 mW/cm². In another example, under the critical condition that the temperature of the attachment surface (i.e., the side of the thermal conductive layer 14 attached to the user's skin) does not exceed 45° C., the driving current of the light irradiation device 1 can be increased to approximately 1.5 A, and the intensity of the emitted light is approximately 18.8 mW/cm². As a result, it can be seen that, by providing the thermal conductive layer 14 in the light irradiation device 1, the light intensity provided by the light irradiation device 1 can be increased from approximately 14.1 mW/cm² to approximately 18.8 mW/cm² under the critical condition that the temperature does not exceed 45° C. Therefore, the light dose provided by the light irradiation device 1 within the same irradiation time can be significantly increased.

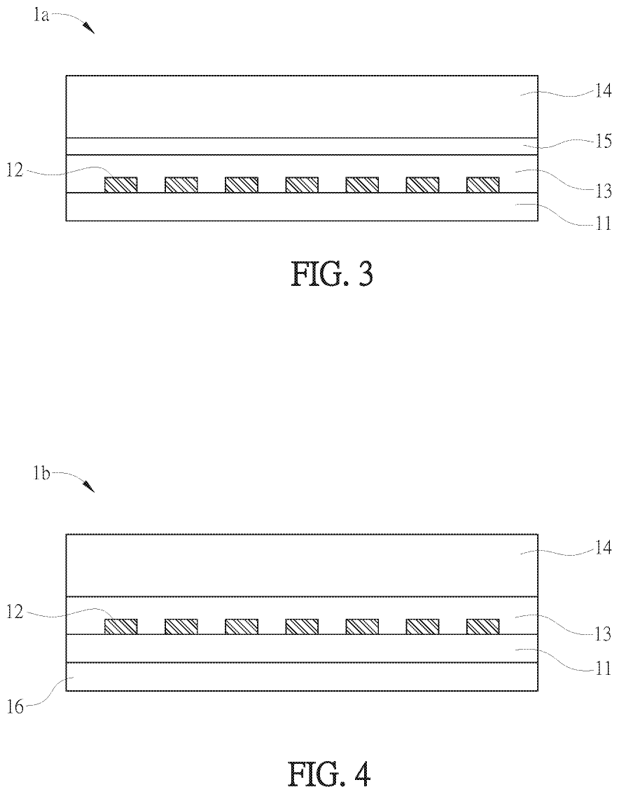

FIG. 3 is a schematic sectional view of a light irradiation device 1a according to a second embodiment of this disclosure.

Unlike the light irradiation device 1 of FIG. 1, the light irradiation device 1a of FIG. 3 further includes a connecting layer 15, which is disposed between the package layer 13 and the thermal conductive layer 14. In this embodiment, the connecting layer 15 can be any suitable adhesive material, such as a thermosetting material, a light-curing material, or other suitable adhesive materials. For example, the connecting layer 15 can be made of a pressure-sensitive adhesive (PSA), an optical clear adhesive (OCA), or an optical clear resin (OCR), etc. In addition, the material of the connecting layer 15 can be, for example, re-adhesive. In this case, the thermal conductive layer 14 can be replaceably disposed on the side of the connecting layer 15 away from the package layer 13. For example, if the concentration of the drug composition contained in the thermal conductive layer 14 decreases with the increase in usage time, a new thermal conductive layer 14 can be provided to replace the used thermal conductive layer 14.

FIG. 4 is a schematic sectional view of a light irradiation device 1b according to a third embodiment of this disclosure.

Unlike the light irradiation device 1 of FIG. 1, the light irradiation device 1b of FIG. 4 further includes a backing layer 16, which is disposed on the side of the substrate 11 away from the light-emitting unit 12. In one embodiment, the backing layer 16 may include a heating element, for example, to provide thermal therapy as needed, thereby further enhancing blood circulation and/or drug absorption. In one embodiment, the backing layer 16 may include a heat dissipation element. In some embodiments, the heat dissipation element may include, for example but is not limited to, a passive heat dissipation element (e.g. a heat sink) or an active heat dissipation element (e.g. a fan). The heat dissipation element is used to provide heat dissipation for the light-emitting unit 12.

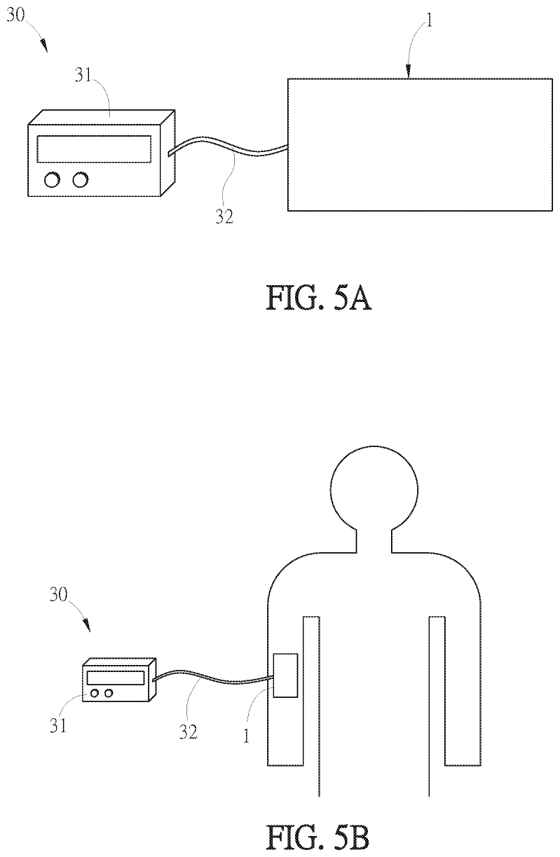

FIG. 5A is a schematic perspective view of the light irradiation apparatus 30 according to an embodiment of this disclosure, and FIG. 5B is a schematic diagram of the light irradiation apparatus 30 of FIG. 5A in use.

As shown in FIG. 5A, the light irradiation apparatus 30 of this embodiment includes a control device 31 and a light irradiation device 1 as shown in FIG. 1. The light irradiation device 1 is electrically connected to the control device 31 through a connecting line 32. The shape of the light irradiation device 1 of this embodiment is rectangular, and the light irradiation device 1 can be designed as, for example, a flexible patch. Other structural designs of the light irradiation device 1 can refer to the aforementioned embodiments, so the detailed description thereof will be omitted. In this case, the control device 31 can transmit a control signal and the electrical energy to the light irradiation device 1 through the connecting line 32 to control and drive the light-emitting unit 12 of the light irradiation device 1 to emit a light. The wavelength of the emitted light is between 650 nm and 830 nm, and the light intensity range of the light is set to 1-200 mW/cm². It should be noted that the light irradiation device 1 can adopt any of the structures of the aforementioned embodiments.

As shown in FIG. 5B, since the shape of the light irradiation device 1 is rectangular, the thermal conductive layer 14 thereof is suitable to be attached to a large affected area, such as an arm (see FIG. 5B) or the back. In this case, the user can operate the control device 31 to transmit a control signal and the electrical energy to the light irradiation device 1 via the connecting line 32 to control and drive the light-emitting unit 12 of the light irradiation device 1 to emit light. The wavelength of the emitted light is between 650 nm and 830 nm, and the light intensity range of the emitted light can be set between 1 mW/cm² and 200 mW/cm² according to different driving currents.

Therefore, the light emitted by the light-emitting unit 12 can pass through the thermal conductive layer 14 and irradiate the affected area, and the heat energy generated by the light-emitting unit 12 can pass through the thermal conductive layer 14 and be transferred to the skin at the affected area. Because the thermal conductive layer 14 has a restricted thermal conductivity, the temperature of the side of the thermal conductive layer 14 that contacts the user's skin can be controlled to be above the body temperature but below a temperature that could cause burns (e.g. below 45° C.). This design also effectively increases the light dose provided by the light irradiation device 1 within the same irradiation time. In this embodiment, the light within this specific wavelength range not only reduces inflammation, speeds wound healing, promotes collagen synthesis, and accelerates angiogenesis, but also stimulates traditional acupuncture points beneath the skin surface. By providing energy to the acupuncture points, the stimulation achieves the effects of traditional acupuncture. Furthermore, the heat energy provided can effectively increase the absorption rate of the pharmaceutical composition contained in the thermal conductive layer 14, thereby enhancing the therapeutic effect.

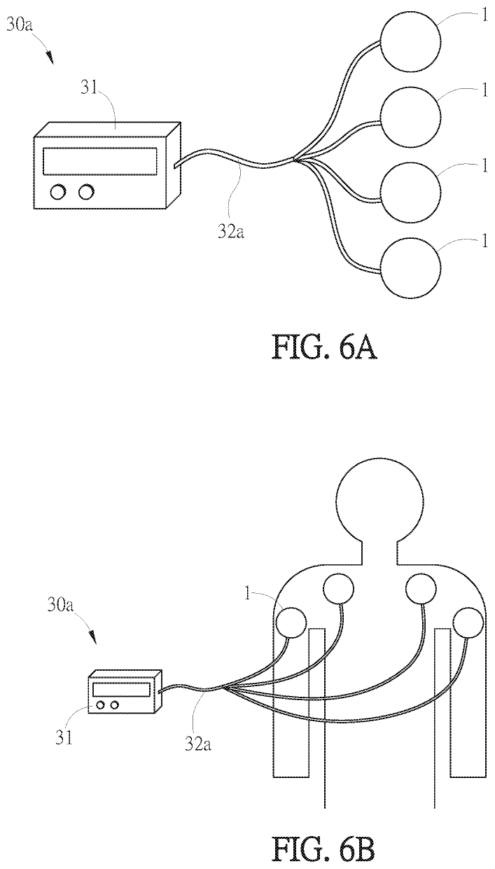

FIG. 6A is a schematic perspective view of a light irradiation apparatus 30a according to another embodiment of this disclosure, and FIG. 6B is a schematic diagram of the light irradiation apparatus 30a of FIG. 6A in use.

Referring to FIG. 6A, the light irradiation apparatus 30a of this embodiment includes a control device 31 and a plurality of light irradiation devices 1. The light irradiation devices 1 are electrically connected to the control device 31 via one or more connecting lines 32a. The shape of the light irradiation devices 1 of this embodiment is circular (e.g. coin-shaped), and the light irradiation devices 1 may be, for example, flexible circular patches. The connecting line 32a may be a one-to-multiple design, allowing one control device 31 to simultaneously connect to multiple light irradiation devices 1. The other structural designs of the light irradiation devices 1 can refer to the aforementioned embodiments, so the detailed description thereof will be omitted. The control device 31 transmits control signals and electrical energy to the multiple light irradiation devices 1 via the connecting line(s) 32a, thereby controlling and driving the light-emitting unit 12 of each light irradiation device 1 to emit light. The emitted light has a wavelength between 650 nm and 830 nm, and the light intensity range of the emitted light is set from 1 mW/cm² to 200 mW/cm².

Referring to FIG. 6B, since the shape of each light irradiation device 1 is circular, the thermal conductive layer 14 thereof is suitable to be attached to the affected area of a single acupuncture point, such as the acupuncture points located on the shoulder, neck, arm, back, etc. In this case, the user can operate the control device 31 to transmit control signals and electrical energy to the multiple light irradiation devices 1 via the connecting line(s) 32a to control and drive the light-emitting unit 12 of each lighting device 1 to emit a light. The wavelength of the emitted light is between 650 nm and 830 nm, and the light intensity range of the emitted light can be set between 1 mW/cm² and 200 mW/cm² according to different driving currents.

In the above embodiments, the light emitted by the light-emitting unit 12 can pass through the thermal conductive layer 14 and irradiate the affected area (or acupuncture point). The heat energy generated by the light-emitting unit 12 can also pass through the thermal conductive layer 14 and be transferred to the skin at the affected area (or acupuncture point). Due to the restricted thermal conductivity of the thermal conductive layer 14, the temperature of the side of the thermal conductive layer 14 that adheres to the user's skin can be controlled to be above the body temperature but below a temperature that could cause burns (e.g. below 45° C.). This design also effectively increases the light dose provided by the light irradiation device 1 within the same irradiation time. In this embodiment, the light within this specific wavelength range not only reduces inflammation, speeds wound healing, promotes collagen synthesis, and accelerates angiogenesis, but also stimulates traditional acupuncture points beneath the skin surface. By providing energy to the acupuncture points, the stimulation achieves the effects of traditional acupuncture. Furthermore, the heat energy provided can effectively increase the absorption rate of the pharmaceutical composition contained in the thermal conductive layer 14, thereby enhancing its therapeutic effect.

Although the disclosure has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the disclosure.

What is claimed is:

1. A light irradiation device, comprising:
a substrate;
a light-emitting unit disposed on the substrate;
a package layer disposed on the light-emitting unit; and
a thermal conductive layer disposed on the package layer, wherein a light transmittance of the thermal conductive layer ranges from 80% to 99%;
wherein, the light-emitting unit is configured to emit a light having a wavelength between 650 nm and 830 nm, and a light intensity range of the light is set from 1 mW/cm² to 200 mW/cm².

2. The light irradiation device of claim 1, wherein a thickness of the thermal conductive layer ranges from 0.05 mm to 5 mm.

3. The light irradiation device of claim 1, wherein a thermal conductivity of the thermal conductive layer ranges from 0.2 W/m-K to 4 W/m-K.

4. The light irradiation device of claim 1, wherein the thermal conductive layer comprises a biocompatible material.

5. The light irradiation device of claim 4, wherein the biocompatible material comprises one or a combination of a hydrogel material and a silicone material.

6. The light irradiation device of claim 1, wherein the light irradiation device is a phototherapy device.

7. The light irradiation device of claim 1, wherein the light irradiation device is a laser acupuncture device.

8. The light irradiation device of claim 1, wherein the thermal conductive layer comprises a pharmaceutical composition.

9. The light irradiation device of claim 8, wherein the pharmaceutical composition comprises one or a combination of a wound dressing and a percutaneous absorption drug.

10. The light irradiation device of claim 1, further comprising:
a connecting layer disposed between the package layer and the thermal conductive layer.

11. A light irradiation apparatus, comprising:
a control device; and
a light irradiation device electrically connected to the control device and comprising:
a substrate;
a light-emitting unit disposed on the substrate;
a package layer disposed on the light-emitting unit; and
a thermal conductive layer disposed on the package layer, wherein a light transmittance of the thermal conductive layer ranges from 80% to 99%;
wherein, the control device controls the light-emitting unit to emit a light having a wavelength between 650 nm and 830 nm, and a light intensity range of the light is set from 1 mW/cm² to 200 mW/cm².

12. The light irradiation apparatus of claim 11, wherein a thickness of the thermal conductive layer ranges from 0.05 mm to 5 mm.

13. The light irradiation apparatus of claim 11, wherein a thermal conductivity of the thermal conductive layer ranges from 0.2 W/m-K to 4 W/m-K.

14. The light irradiation apparatus of claim 11, wherein the thermal conductive layer comprises a biocompatible material.

15. The light irradiation apparatus of claim 14, wherein the biocompatible material comprises one or a combination of a hydrogel material and a silicone material.

16. The light irradiation apparatus of claim 11, wherein the light irradiation device is a phototherapy device.

17. The light irradiation apparatus of claim 11, wherein the light irradiation device is a laser acupuncture device.

18. The light irradiation apparatus of claim 11, wherein the thermal conductive layer comprises a pharmaceutical composition.

19. The light irradiation apparatus of claim 18, wherein the pharmaceutical composition comprises one or a combination of a wound dressing and a percutaneous absorption drug.

20. The light irradiation apparatus of claim 11, wherein the light irradiation device further comprises:
a connecting layer disposed between the package layer and the thermal conductive layer.
